(19) United States
(12) Patent Application Publication
(10) Pub. No.: US 2026/0153511 A1
(43) Pub. Date: Jun. 4, 2026

(54) LACTYLATED NIJMEGEN BREAKAGE SYNDROME 1 PROTEIN AND USES THEREOF

(71) Applicant: THE SEVENTH AFFILIATED HOSPITAL, SUN YAT-SEN UNIVERSITY, Shenzhen (CN)

(72) Inventors: Changhua Zhang, Shenzhen (CN); Hengxing Chen, Shenzhen (CN)

(21) Appl. No.: 19/529,201

(22) Filed: Feb. 4, 2026

Related U.S. Application Data
(63) Continuation-in-part of application No. PCT/CN2024/116986, filed on Sep. 4, 2024.

(30) Foreign Application Priority Data
Apr. 3, 2024 (CN) 202410402099.1

(51) Int. Cl. G01N 33/575; G01N 33/68

(52) U.S. Cl. CPC G01N 33/57595; G01N 33/6875; G01N 2440/00

(57) ABSTRACT

Disclosed are a lactylated NBS1 protein and uses thereof. A lactylation site in this application is lysine at position 388 of NBS1 protein. Lactylation at K388 site of the NBS1 protein activates the DNA repair pathway and has multiple functions, including promoting radiotherapy and chemotherapy resistance in tumor cells and enhancing homologous recombination in DNA repair. By targeted inhibition of the lactylated NBS1 protein, tumor resistance to radiotherapy or chemotherapy can be reversed, providing a potential target for reversing tumor resistance to radiotherapy or chemotherapy.

### CROSS-REFERENCE TO RELATED APPLICATIONS

The present application is a Continuation-in-part Application of PCT application No. PCT/CN2024/116986 filed on September 4, 2024, which claims the benefit of Chinese Patent Application No. 202410402099.1 filed on April 3, 2024. The contents of the above-identified applications are hereby incorporated by reference in their entirety.

### REFERENCE TO SEQUENCE LISTING

This application includes a Sequence Listing filed electronically as an XML file named “ US2501174H-PCT_SL.xml”, created on December 25, 2025, with a size of 18,755 bytes. The Sequence Listing is incorporated herein by reference.

### TECHNICAL FIELD

The present application relates to the field of tumor radiotherapy, and in particular to a lactylated Nijmegen breakage syndrome 1 (NBS1) protein and uses thereof.

### BACKGROUND

A DNA double-strand break (DSB) is considered the most serious form of DNA damage because an unrepaired double-strand break is sufficient to cause permanent growth arrest or cell death. When a double-strand break occurs in a cell's DNA, the cell immediately initiates DNA repair in order to survive and maintain DNA integrity. An MRE11-RAD50-NBS1 (MRN) complex plays a crucial role in sensing and repairing of DNA damage. When a double-strand break occurs in DNA, the MRN complex forms immediately and is rapidly recruited to a DNA damage site, initiating DNA repair. The MRN complex is composed of MRE11 (MRE11 homolog, a double strand break repair nuclease), RAD50 (RAD50 double strand break repair protein), and NBS1 (Nijmegen breakage syndrome 1 protein). MRE11 and RAD50 proteins are responsible for cleaving broken DNA. The NBS1 protein is responsible for regulating functions of MRE11 and RAD50. Therefore, the NBS1 protein is a regulatory factor of the MRN complex. The MRN complex can maintain genome stability and prevent malignant transformation of normal cells. The MRN complex’s function in cancer development and the MRN complex’s potential as an anti-cancer target have been extensively explored in various types of cancer. Analysis of gastric cancer specimens surgically resected after chemotherapy has shown that low expression levels of the MRN complex are associated with a strong response to chemotherapy and surgical resection. In vitro experiments have shown that with increasing DNA damage and cytotoxicity, disruption of the MRN complex or its components can make cancer cells more sensitive to cisplatin.

NBS1 is a member of the MRN complex and a regulatory factor of the MRN complex, responsible for regulating the function of the MRN complex. NBS1 mutation is a main cause of Nijmegen breakage syndrome (NBS). Symptoms of NBS patients include slow development and a high susceptibility to cancer. In tumorigenesis studies, NBS1 mutation has been found to be associated with increased cancer susceptibility in breast cancer, prostate cancer, lung cancer, liver cancer, and intrahepatic cholangiocarcinoma. In studies on chemotherapy resistance mechanisms, downregulation of NBS1 expression is associated with hypoxia-induced chemotherapy resistance, inhibition of DSB repair pathways, and p53 activation in medulloblastoma cells. In studies on radiotherapy resistance mechanisms, NBS1 can stabilize hypoxia-inducible factors and promote cancer cell migration and invasion under ionizing radiation (IR).

Protein lactylation plays a key role in human physiological and pathological processes. Given the important role of NBS1 in occurrence and development of tumors, further research on the lactylation of the NBS1 protein is of great significance.

### SUMMARY

The objective of this application is to overcome shortcomings of the prior art by providing a lactylated NBS1 protein and uses thereof. Lactylation of NBS1 protein plays an important role in DNA damage repair. Targeted inhibition of lactylation of the NBS1 protein can reverse tumor resistance to radiotherapy or chemotherapy.

To achieve the above objective, technical solutions adopted in this application include:

In a first aspect, this application provides use of a reagent for detecting a lactylation level of NBS1 protein in the preparation of a kit for predicting efficacy of radiotherapy or chemotherapy for a tumor, where a lactylation site is lysine at position 388 of the NBS1 protein.

Through lactylation proteomics, the applicant has discovered that lysine (K) at position 388 of the NBS1 protein is a lactylated amino acid. The lactylated NBS1 protein can promote MRN complex formation, thereby contributing to tumor resistance to radiotherapy or chemotherapy. Statistical analysis of immunohistochemistry data shows that lactylation levels of the NBS1 protein in tumor tissues of chemotherapy-resistant gastric cancer are significantly higher than those in tumor tissues of gastric cancer sensitive to chemotherapy. Therefore, the higher the lactylation level of the NBS1 protein, the worse the efficacy of chemotherapy and/or radiotherapy in a tumor patient.

In some examples, a lactylation level of the NBS1 protein is regulated by a lysine lactyltransferase , e.g., Tat-interactive protein 60 (TIP60).

In this application, a relationship between the lysine lactyltransferase TIP60 and the NBS1 protein is confirmed using co-immunoprecipitation and Western blotting techniques. Overexpression of the lysine lactyltransferase TIP60 can promote the lactylation level of the NBS1 protein; and knockdown of the lysine acetyltransferase TIP60 can reduce the lactylation level of the NBS1 protein. This indicates that the lysine acetyltransferase TIP60 can interact with the NBS1 protein, and TIP60 can regulate the lactylation of the NBS1 protein.

In some examples, the higher the lactylation level of the NBS1 protein, the worse the efficacy of chemotherapy and/or radiotherapy in a tumor patient.

In a second aspect, the present application provides use of lactylated NBS1 protein as a target in the preparation of a drug for reversing tumor resistance to radiotherapy or chemotherapy, where a lactylation site is lysine at position 388 of the NBS1protein.

In a third aspect, this application provides a pharmaceutical composition for reversing tumor resistance to radiotherapy or chemotherapy, including an agent that specifically inhibits lactylation of NBS1 protein.

In this application, a drug sensitivity assay by cell counting kit-8 (CCK8) and a flow cytometry-based apoptosis assay demonstrate that abolishing lactylation of the NBS1 protein can reverse tumor cell resistance to radiotherapy and chemotherapy.

In some examples, the agent is selected from the group consisting of a small molecule inhibitor, a nucleic acid, and a polypeptide.

In some examples, the pharmaceutical composition further includes a pharmaceutically acceptable carrier.

In a fourth aspect, the present application provides use of the above-mentioned pharmaceutical composition in the preparation of a drug for reversing tumor resistance to radiotherapy or chemotherapy. Further, this application provides a method for reversing tumor resistance to radiotherapy or chemotherapy, including administering a therapeutically effective amount of the pharmaceutical composition to a tumor patient exhibiting tumor resistance to radiotherapy or chemotherapy.

In a fifth aspect, the present application provides a kit for predicting efficacy of radiotherapy or chemotherapy for a tumor, including a reagent for detecting a lactylation level of NBS1 protein, where a lactylation site is lysine at position 388 of the NBS1 protein. In some examples, lactylation of the NBS1 protein is binding of a lactyl group to an amino group via an amide bond, and the amino group is located at a side chain end of the lysine at position 388 of the NBS1 protein. “Lactyl group” is the remaining part of a lactic acid molecule after losing a hydroxyl group (-OH), with a chemical formula CH₂CH₂CO-. In biochemistry, a lactyl group can modify a lysine residue in a protein through a covalent bond, thereby forming lactylation.

In a sixth aspect, the present application provides a gastric cancer cell line AGS including in its genome a mutant NBS1 gene, where the mutant NBS1 gene includes a mutation when compared to wild-type NBS1 gene, and the mutation changes the codon for lysine at position 388 of a protein encoded by the wild-type NBS1 gene to a codon for an amino acid that cannot be lactylated. In some examples, the amino acid that cannot be lactylated is arginine.

Beneficial effects of this application include:

In this application, lactylation proteomics reveals that lysine at position 388 of the NBS1 amino acid sequence undergoes lactylation. Lactylation of the NBS1 protein plays an important role in DNA damage repair. The lactylated NBS1 protein can promote MRN complex formation, activate DNA repair pathways, and significantly enhance tumor resistance to radiotherapy and chemotherapy.

The lactylated NBS1 protein can serve as a biomarker to predict efficacy of radiotherapy or chemotherapy in a tumor patient. By targeted inhibition of lactylation of the NBS1 protein, tumor resistance to radiotherapy or chemotherapy can be reversed.

### BRIEF DESCRIPTION OF THE DRAWINGS

FIG. 1 is a structural diagram of the NBS1 protein, where “FHA” represents forehead-associated domain, “BRCT1” represents breast cancer C-terminal domain 1, “BRCT2” represents breast cancer C-terminal domain 2, and “MBD” represents Mre11 binding domain.

FIG. 2 shows mass spectrometry results of the NBS1 protein, where “NBS1-K388la” represents lactylation of the lysine at position 388 of the NBS1 protein.

FIG. 3A, FIG. 3 B, and FIG. 3C show results of interaction between the lysine lactyltransferase TIP60 and the NBS1 protein, where FIG. 3A shows results of an endogenous immunoprecipitation experiment, which illustrates a location of an IgG light chain. In FIG. 3A, “Input” represents a total protein lysate extracted from cells before immunoprecipitation; “IP: NBS1” refers to immunoprecipitation using a specific antibody against the NBS1 protein; and “IgG” serves as a negative control antibody, and the IgG antibody consists of two heavy chains and two light chains.

FIG. 3B shows Western blot results of TIP60 overexpression. In FIG. 3B, “-” indicates that AGS cells do not contain an exogenous plasmid Flag-TIP60 (i.e., plasmid PLVX-Flag-TIP60 in Example 2); “+” indicates that AGS cells contain the exogenous plasmid Flag-TIP60; and “Flag” represents the Flag protein. “NBS1-K388la” represents expression of lactylation at position K388 of the NBS1 protein, “β-tubulin” and “H4” represent internal controls, and “H4K8ac” represents a positive control.

FIG. 3C shows Western blot results of TIP60 knockdown. In FIG. 3C, “-1” and “-2” correspond to two different siRNA sequences of TIP60 respectively, “NC” represents a negative control, “Si-TIP60” represents Si-TIP60 RNA, “NBS1-K388la” represents lactation at position K388 of the NBS1 protein, “β-tubulin” and “H4” represent internal controls, and “H4K8ac” represents a positive control.

FIG. 4A and FIG. 4B show that lactylation of the NBS1 protein promotes MRN complex formation. FIG. 4A shows results of first-generation sequencing; and FIG. 4B shows results of Western blot experiments, where “NBS1-K388la” represents lactylation at position K388 of the NBS1 protein, “IP” represents immunoprecipitation, “AGS” represents AGS cells, “Parental” represents AGS parental cells, “NBS1-K388R” indicates that the lysine (K) at position 388 of NBS1 is mutated to arginine (R), “Input” represents a total protein lysate extracted from cells before immunoprecipitation, “IP: NBS1” refers to immunoprecipitation using a specific antibody against the NBS1 protein, “β-tubulin” represents an internal control, and “ATM” (ataxia-telangiectasia mutated protein) and “p-ATM (S1981)” represent positive controls.

FIG. 5A, FIG. 5 B, and FIG. 5C show results of reversing radiotherapy and chemotherapy resistance in tumor cells by abolishing lactylation of the NBS1 protein. FIG. 5A shows experimental results of cell counting kit-8 (CCK8), where “Cell viability” represents cell survival rate; FIG. 5B shows results of a flow cytometry-based apoptosis assay, where “Annexin V+ cells” refers to cells that are detected to contain Annexin V; and FIG. 5C shows experimental results of CCK8.

FIG. 6A and FIG. 6B show a relationship between high level of NBS1 lactylation and efficacy of chemotherapy in a tumor patient. FIG. 6A shows results of immunohistochemical staining; and FIG. 6B shows statistical results of immunohistochemical data.

### DETAILED DESCRIPTION

In order to better illustrate this application, numerous specific details are provided in the following detailed examples. Those skilled in the art shall understand that this application can be implemented even without certain specific details. In order to highlight the subject matter of this application, methods, means, equipment, and steps well known to those skilled in the art have not been described in detail in other examples.

To better illustrate the purpose, technical solution, and advantages of this application, the following will provide further explanation of this application in conjunction with specific examples.

In examples of this application, experimental steps of Western blot include the following:

1. Sample preparation

Cell lysis: Cells were lysed in a lysis buffer, and a protease inhibitor and a phosphatase inhibitor were then added to a resulting mixture to prevent protein degradation and dephosphorylation.

Protein concentration determination: A total protein concentration in a sample was determined using a bicinchoninic acid (BCA) kit to facilitate subsequent equal mass loading.

2. Protein denaturation: The sample was heated in a 95-100 °C water bath for 5-10 minutes to denature proteins.

3. Sodium dodecyl sulfate‐polyacrylamide gel electrophoresis (SDS-PAGE)

Preparing a gel: A appropriate gel concentration was selected based on a molecular weight of a target protein to prepare a resolving gel and a stacking gel.

Loading: A denatured protein sample was added to a gel well, along with a protein molecular weight marker (MWM).

Electrophoresis: The power was turned on, and an appropriate voltage was set until bromophenol blue indicator reached a bottom of the gel, then electrophoresis was stopped.

4. Membrane transfer

Preparing a membrane transfer device: The gel and a poly(vinylidene fluoride) (PVDF) membrane were soaked in a transfer buffer.

Assembly of a transfer clamp: A first sponge, a first filter paper, the gel, the PVDF membrane, a second filter paper, and a second sponge were assembled in sequence, ensuring that there were no air bubbles.

Membrane transfer: The power was turned on, and an appropriate voltage and time were set to transfer proteins from the gel to the PVDF membrane.

5. Blocking: The transferred PVDF membrane was placed in a solution containing 5% (5g/100 ml) skim milk powder and incubated at room temperature for 1 hour.

6. Antibody incubation

Primary antibody incubation: The PVDF membrane was placed in a diluted primary antibody solution and incubated overnight at 4 °C.

Washing: A resulting PVDF membrane was washed three times with a tris buffered saline with Tween® 20 (TBST) buffer, for five minutes each time.

Secondary antibody incubation: A resulting PVDF membrane was placed in a diluted secondary antibody solution and incubated at room temperature for 1 hour.

Second washing: A resulting PVDF membrane was washed three times with a tris buffered saline with Tween® 20 (TBST) buffer, for five minutes each time.

7. Color development

Color development via electrochemiluminescence (ECL): A resulting PVDF membrane was placed in an ECL luminescent solution and incubated for 1-2 minutes, then exposed in a chemiluminescence imager to record a signal.

8. Result analysis

Band analysis: Based on a position and intensity of a band, an expression level and a molecular weight of a target protein were determined.

Example 1

One million AGS cells were collected, then lysed using a radioimmunoprecipitation assay (RIPA) lysis buffer, and intracellular proteins were extracted. Then, trypsin was used to cleave the extracted proteins into peptides. Lactylated peptides were enriched using the lactylation antibody Anti-L-Lactyl Lysine Rabbit pAb (purchased from Jingjie PTM BioLab (HangZhou) Co., Inc., catalog number: PTM-1401), followed by mass spectrometry detection.

Steps of the mass spectrometry detection included the following:

In liquid chromatography-tandem mass spectrometry (LC–MS/MS) analysis, trypsin-digested peptides were dissolved in solvent A (an aqueous solution containing 2% (v/v) acetonitrile and 0.1% (v/v) formic acid). A resulting mixture was then loaded onto a reversed-phase column. Peptides were separated by gradient elution under the following conditions: a concentration of solvent B (an acetonitrile solution containing 0.1% (v/v) formic acid) was increased from 6% to 24% (v/v) over 70 minutes, then from 24% to 35% over 14 minutes, then from 35% to 80% over 3 minutes, and finally held at 80% for 3 minutes. The entire process was performed at a constant flow rate of 450 nL/min using a nanoElute ultra-high performance liquid chromatography system from BRUKER DALTONICS INC. The peptides were subjected to capillary source and analyzed using a timsTOF Pro mass spectrometer from BRUKER DALTONICS INC. The timsTOF Pro mass spectrometer was operated in a parallel accumulation-serial fragmentation (PASEF) mode. Precursor ions and fragment ions were analyzed on a time-of-flight (TOF) detector (MS/MS scan ranged from 100 to 1700 m/z). The dynamic exclusion time was 30 seconds. Precursor ions with charge states from 0 to 5 were selected for fragmentation. Ten PASEF-MS/MS scans were acquired in each cycle. An applied electrospray voltage was 1.75 kV.

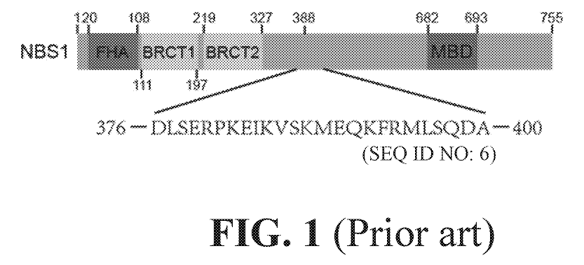

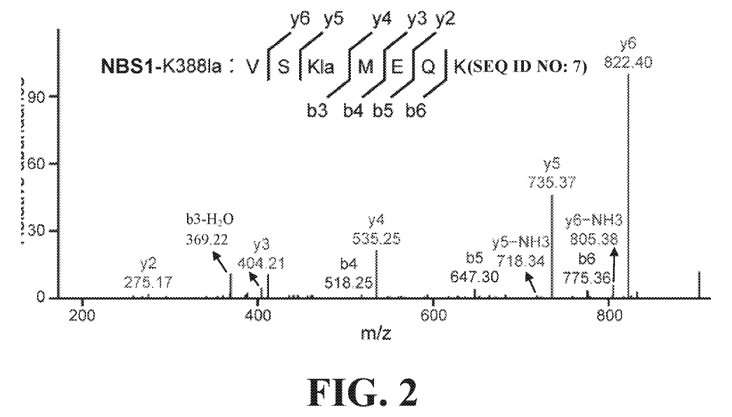

A structure of the NBS1 protein is shown in FIG. 1. Results of mass spectrometry detection are shown in FIG. 2. The results show that lysine at position 388 of the NBS1 protein contains lactylation.

Example 2

1. A relationship between the lysine lactyltransferase TIP60 and the NBS1 protein was confirmed using co-immunoprecipitation and Western blotting techniques.

Specific steps included the following:

(1) One million 293T cells were collected and lysed with 1 mL of a cell lysis buffer. A resulting mixture was allowed to stand on ice for 30 min to lyse, with vortexing every 15 min during this period. A resulting lysate was then centrifuged at 12,000 g and 4 °C for 20 minutes, a resulting pellet was discarded, and a resulting supernatant was collected.

(2) 2 μg of a corresponding NBS1 antibody (purchased from Cell Signaling Technology, Inc., catalog number: 14956) was added to the resulting supernatant, and a mixture obtained was incubated in a shaker at 4 °C for 2 hours to obtain a incubation solution.

(3) 20 μL of protein A/G-beads (magnetic beads) were added to a 1.5 mL centrifuge tube, and the protein A/G-beads were washed with 800 μL of RIPA lysis buffer. The supernatant obtained from washing was discarded, and then the washing was repeated three times. The washed magnetic beads were added to the incubation solution prepared in step (2), and a resulting mixture was incubated overnight in a shaker at 4 °C.

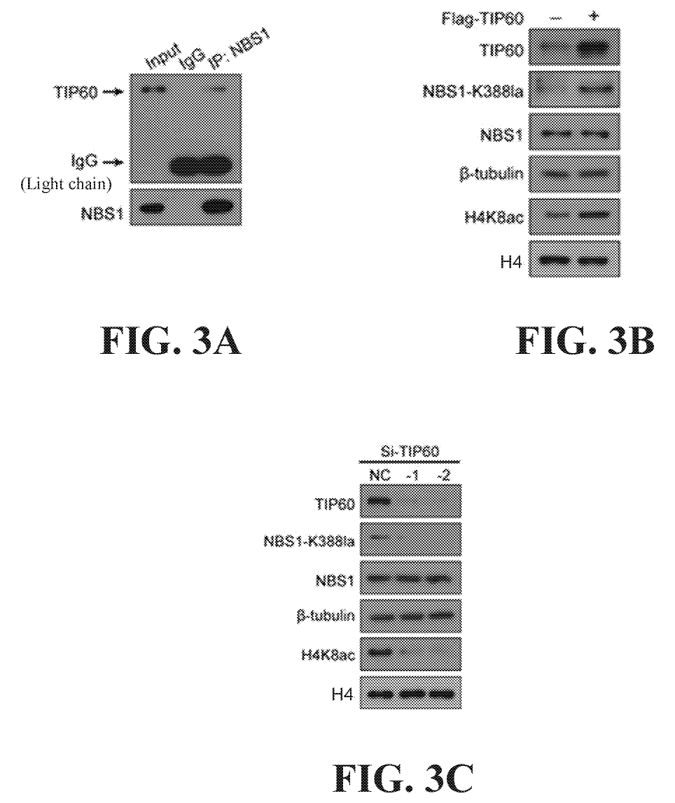

(4) The next day, the centrifuge tube was placed on a magnetic rack. After the magnetic beads accumulated on the wall of the centrifuge tube, supernatant in the centrifuge tube was carefully aspirated, and then resulting magnetic beads were washed with 800 μL of RIPA lysis buffer, with the washing repeated for 5 times. 80 μL of RIPA lysis buffer and 20 μL of 5× sodium dodecyl sulfate (SDS) loading buffer were added to the washed centrifuge tube, which was then shaken until substances therein were thoroughly mixed. A resulting centrifuge tube was heated at 99 °C for 10 min to denature proteins therein. A relationship between TIP60 protein (an antibody against TIP60 was purchased from Proteintech Group, Inc., with a catalog number: 10827-1-AP) and the NBS1 protein (an antibody against NBS1 was purchased from Cell Signaling Technology, Inc., with a catalog number: 14956) was detected by Western blot. Results are shown in FIG. 3A. The IgG (light chain) antibody was purchased from Cell Signaling Technology (CST), Inc., with a catalog number of 5742.

Endogenous immunoprecipitation experiments demonstrated that the lysine lactyltransferase TIP60 interacts with the NBS1 protein.

2. To further confirm the relationship between the lysine lactyltransferase TIP60 and lactylation of the NBS1 protein, the lysine lactyltransferase TIP60 was overexpressed and knocked down, respectively.

Specific experimental methods included the following:

(1) Overexpression of the lysine lactyltransferase TIP60: Overexpression plasmids PLVX-Flag-TIP60 (SEQ ID NO: 5, constructed by Guangzhou IGE Biotechnology Co., Ltd.) were transfected into AGS cells. Twenty-four hours after transfection, proteins were collected for Western blot experiments. An antibody against TIP60 was purchased from Proteintech Group, Inc., with a catalog number of 10827-1-AP. An antibody against NBS1-K388la was purchased from Jingjie PTM BioLab (HangZhou) Co., Inc. An antibody against NBS1 was purchased from Cell Signaling Technology, Inc., with a catalog number of 14956. An antibody against β-tubulin was purchased from Proteintech Group, Inc., with a catalog number of 10068-1-AP. An antibody against H4K8ac was purchased from PTM BIO LLC, with a catalog number of PTM-120. An antibody against H4 was purchased from Abcam Limited, with a catalog number of ab31830. Results are shown in FIG. 3 B.

(2) Knockdown of the lysine lactyltransferase TIP60: TIP60 was knocked down by transfecting Si-TIP60 RNA (siRNA targeting TIP60) (RNA1: ACGGAAGGUGGAGGUGGUU, SEQ ID NO: 1; RNA2: AAGAAGAUCCAGUUCCCCAAGUU, SEQ ID NO: 2; and the two RNA strands involved in an experiment of siRNA knockdown were two guide strands with different sequences) into AGS cells. Western blot experiments were then performed. Results are shown in FIG. 3 C.

Experimental results demonstrated that overexpression of the lysine lactyltransferase TIP60 can increase the expression of lactylation of the NBS1 protein; and knockdown of the lysine lactyltransferase TIP60 can reduce the lactylation level of NBS1 protein.

In summary, the lysine lactyltransferase TIP60 can interact with the NBS1 protein, and TIP60 can regulate the lactylation of the NBS1 protein.

Example 3

1. Genome editing and immunoprecipitation techniques were used to determine whether lactylation of the NBS1 protein could promote MRN complex formation.

An experimental procedure included the following:

The lysine at position 388 of a protein encoded by the NBS1 gene in the genome of a AGS cell was mutated to arginine, and thus a genome editing cell line AGS-NBS1-K388R (i.e., AGS NBS1-K388R genome editing cells) was constructed, thereby abolishing the lactylation of the NBS1 protein.

A construction method for the genome editing cell line AGS-NBS1-K388R included the following:

(1) First, a spacer DNA sequence and an extended DNA sequence were synthesized. The spacer DNA sequence was 5’-GAAATCAAAGTCTCCAAAA-3’ (SEQ ID NO: 3); and the extended DNA sequence was 5’-TTTTTGTTCCATTCTGGAGACTTTGAT-3’ (SEQ ID NO: 4).

(2) Using a method of Golden Gate assembly, a plasmid pU6-pegRNA-GG (purchased from ADDGENE INC., #132777), the spacer DNA sequence, and the extended DNA sequence were ligated into a single plasmid pU6-pegRNA-GG-NBS1-K388R.

(3) A plasmid PCMV-PE2 (purchased from ADDGENE INC., #132775) and the plasmid pU6-pegRNA-GG-NBS1-K388R were transfected into the gastric cancer cell line AGS (purchased from Shanghai Cell Bank of Chinese Academy of Sciences, #TCHu232).

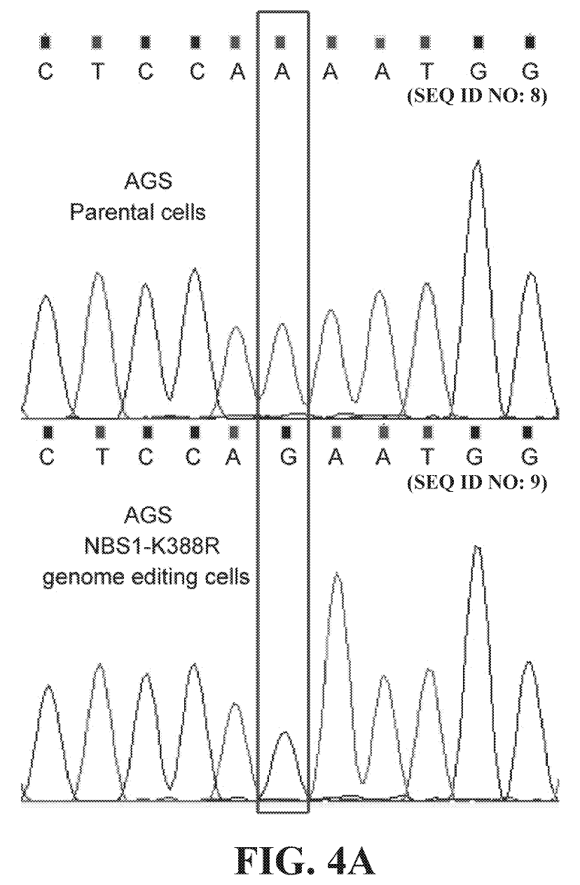

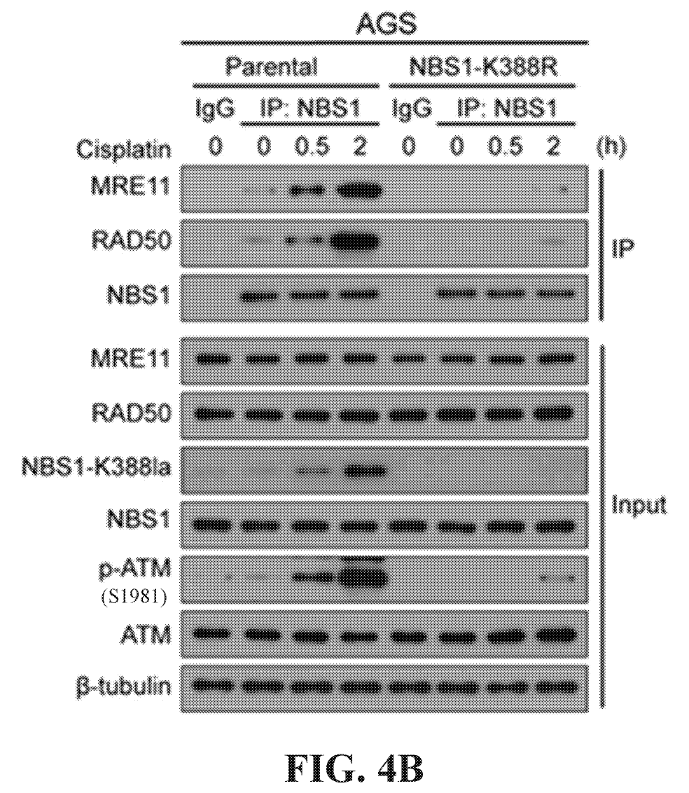

(4) Single clones of transfected AGS cells were selected and sequenced. Sequencing results are shown in FIG. 4A.

First-generation sequencing results showed that the genome editing cell line AGS-NBS1-K388R was successfully constructed in this application.

2. Investigating a relationship between lactylation of the NBS1 protein and MRN complex formation

As verified by immunoprecipitation (experimental steps as shown in Example 2) and Western blot experiments (FIG. 4B), in AGS parental cells, protein expression levels of the MRN complex (meiotic recombination 11 (MRE11), RAD50, and NBS1) increase with increase of NBS1-K388la levels. Therefore, it can be concluded that lactylation of the NBS1 protein can promote MRN complex formation. However, compared to AGS parental cells, an NBS1-K388R mutant cell line (which abolished lactylation of the NBS1 protein after mutation) significantly reduces protein expression levels of the MRN complex (MRE11, RAD50, and NBS1). From this, it can be concluded that abolishing the lactylation of the NBS1 protein can significantly reduce the formation of the MRN complex. Therefore, it can be concluded from FIG. 4B that lactylation of the NBS1 protein promotes the MRN complex formation, while abolishing the lactylation of the NBS1 protein significantly reduces the MRN complex formation (FIG. 4B). In FIG. 4B, an antibody against RAD50 was purchased from Becton, Dickinson and Company (BD), with a catalog number of 611010; an antibody against MRE11 was purchased from Abcam Limited, with a catalog number of ab33125; an antibody against p-ATM (S1981) was purchased from Cell Signaling Technology (CST), Inc., with a catalog number of 13050; and an antibody against ATM was purchased from Cell Signaling Technology (CST), Inc., with a catalog number of 2873.

Example 4

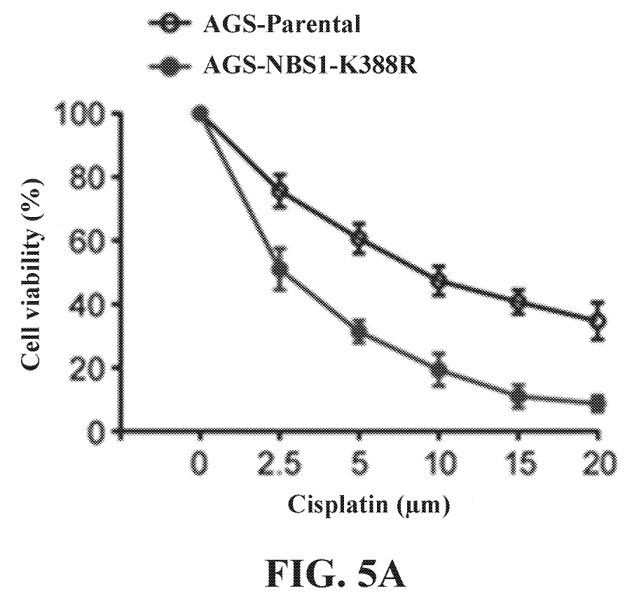

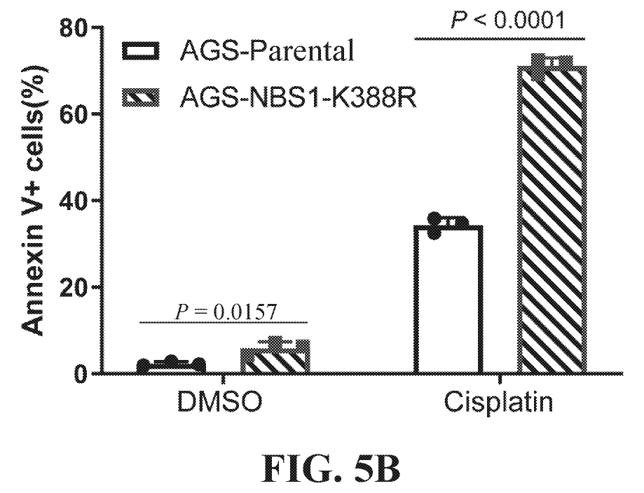

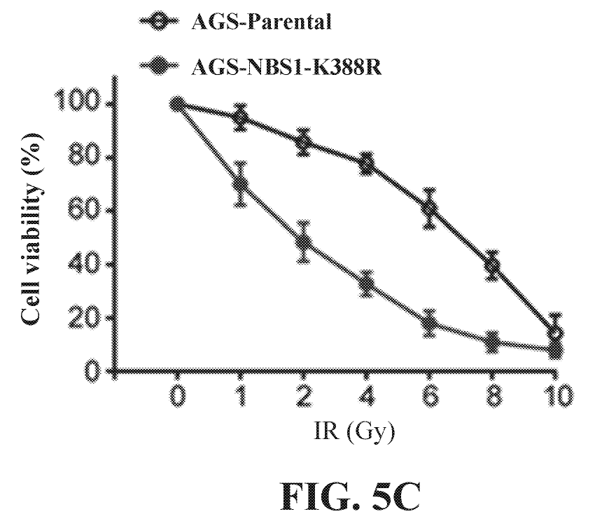

A drug sensitivity assay and a flow cytometry-based apoptosis assay were performed on the genome editing cell line AGS-NBS1-K388R and a parental cell line of AGS using a CCK-8 kit (HANGZHOU FUDE BIOLOGICAL CO., LTD., catalog number: FD378) and an Annexin V-FITC flow cytometry apoptosis kit (Abcam Trading (Shanghai) Co., Ltd., catalog number: ab14085). Results are shown in FIG. 5 A, FIG. 5B, and FIG. 5C.

The drug sensitivity assay included the following steps (refer to an operation guide in the CCK-8 kit):

(1) 100 µL of cell suspension was prepared in a 96-well plate, then the 96-well plate was pre-incubated in an incubator for 24 hours (at 37 °C with 5% (v/v) CO₂);

(2) 10 µL of cisplatin at different concentrations was added to the 96-well plate, which was then incubated in the incubator for an appropriate period of time;

(3) 10 µL of CCK-8 solution was added to each well of the 96-well plate;

(4) The 96-well plate was incubated in the incubator for 2 hours;

(5) Absorbance of each well in the 96-well plate at 450 nm was measured using a microplate reader.

The flow cytometry-based apoptosis assay included the following steps (refer to an operation guide in the Annexin V-FITC flow cytometry apoptosis kit):

Cells were incubated with Annexin V-FITC, including the following steps:

(a) 1-5 × 10⁵ cells were collected by centrifugation;

(b) Resulting cells were resuspended in 500 μL of binding buffer II;

(c) 5 μL of Annexin V-FITC II and 5 μL of propidium iodide II were added to a resulting mixture;

(d) The obtained cells were incubated at room temperature in the dark for 5 minutes, and then subjected to flow cytometry detection.

The flow cytometry detection included the following steps:

Binding of Annexin V-FITC was analyzed by flow cytometry using a FITC detector (usually FL1) (excitation wavelength = 488 nm; emission wavelength = 530 nm). Results of propidium iodide (PI) staining were analyzed using an emission signal detector (usually FL2) for phycoerythrin. Adherent cells were gently digested and washed once with a serum-containing medium before incubation with Annexin V-FITC II.

Results of CCK8 assays and flow cytometry-based apoptosis assays demonstrated that when treated with a chemotherapeutic drug cisplatin, an NBS1-K388R mutant cell line (i.e., AGS-NBS1-K388R) (abolishing lactylation of the NBS1 protein) exhibited lower cell viability when compared to parental cells (i.e., AGS-Parental) (FIG. 5A) and higher levels of apoptosis when compared to parental cells (FIG. 5B). Therefore, it can be concluded that abolishing the lactylation of the NBS1 protein can reverse chemotherapy resistance. Results of CCK8 assays showed that in the NBS1-K388R mutant cell line (abolishing lactylation of the NBS1 protein), cell viability was lower than that of parental cells when exposed to ionizing radiation (IR). Therefore, it can be concluded that abolishing lactylation of the NBS1 protein can reverse radiotherapy resistance (FIG. 5C).

Therefore, this example demonstrated through drug sensitivity assays by CCK8 assays and flow cytometry-based apoptosis assays that abolishing lactylation of the NBS1 protein can reverse tumor cell resistance to radiotherapy and chemotherapy.

Example 5

In this example, tumor tissue samples from 94 cases of gastric cancer before neoadjuvant chemotherapy were collected from the Digestive Medicine Center of the Seventh Affiliated Hospital of Sun Yat-sen University. According to principles of tumor regression grade (TRG), these 94 patient samples were divided into two groups: chemotherapy-sensitive (Sensitive) and chemotherapy-resistant (Resistant):

Chemotherapy-sensitive (Sensitive) group: TRG grades 0-2 were defined as chemotherapy-sensitive;

Chemotherapy-resistant (Resistant) group: TRG grade 3 was defined as chemotherapy-resistant.

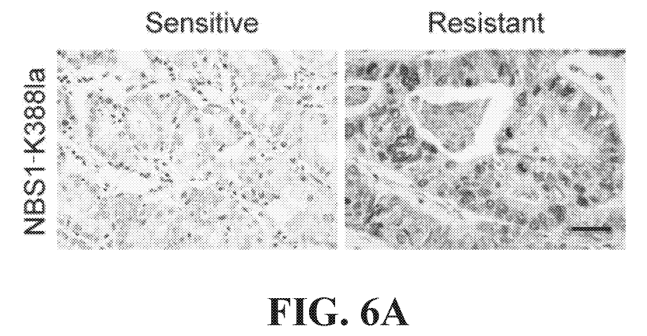

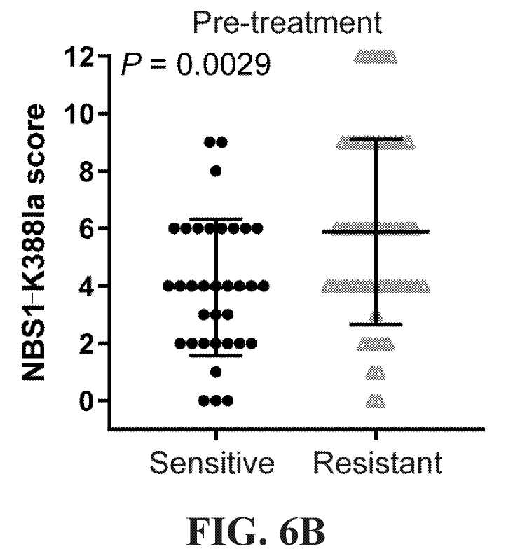

Subsequently, immunohistochemical staining was performed using the antibody against NBS1-K388la (this antibody can specifically recognize lactylation of the NBS1 protein). Results of immunohistochemistry (IHC) are shown in FIG. 6A.

In this example, an immunohistochemistry (IHC) experiment included the following steps:

(1) Tissue fixation: A tissue sample was soaked in a fixative, and a fixation time was usually 12-24 hours.

(2) Tissue dehydration and embedding. Dehydration: The fixed tissue was sequentially soaked in ethanol at different concentrations (70%, 80%, 90%, 95%, and 100% (v/v)) for 15-30 minutes each time to remove water. Transparency: A dehydrated tissue was soaked in xylene to make it transparent, facilitating paraffin embedding. Embedding: A transparent tissue was placed in melted paraffin; after the paraffin solidified, a tissue block was made.

(3) Sectioning: A tissue block was sliced into thin sections using a microtome (typically, the thickness was 4-5 microns). Pasting a section: A section was pasted on a treated slide (a slide was typically treated with polylysine or gelatin) to prevent the section from falling off. Baking the section: The slide was placed in an oven and baked at 60-65 °C for 1-2 hours to ensure the section adhered firmly to the slide.

(4) Dewaxing and hydration. Dewaxing: A resulting section was soaked in xylene to remove the paraffin, 10-15 minutes each time, usually 2-3 times. Hydration: A resulting section was sequentially soaked in ethanol at different concentrations (from 100% to 70% (v/v)), and finally rinsed with distilled water to restore a hydrated state of the section.

(5) Antigen Retrieval: A resulting section was placed in a container containing a retrieval solution (citrate buffer, pH 6.0); a resulting container was then heated in a microwave or pressure cooker until boiling, which was kept for 10-20 minutes; then the heated container was allowed to cool naturally.

(6) Blocking: A resulting section was soaked in a blocking solution and incubated at room temperature for 30 minutes.

(7) Incubation with a primary antibody: A resulting section was placed in an diluted primary antibody solution (diluted according to an antibody manual, usually at a dilution of 1:50–1:200) and incubated overnight at 4 °C.

(8) A resulting section was washed with PBS three times, 5 minutes each time.

(9) Incubation with a secondary antibody: A resulting section was placed in a diluted secondary antibody solution (usually at a dilution of 1:200-1:1000) and incubated at room temperature for 30 minutes to 1 hour.

(10) Washing: A resulting section was washed with PBS three times, 5 minutes each.

(11) Diaminobenzidine (DAB) staining: A resulting section was placed in a DAB staining solution and incubated at room temperature for 3–10 minutes until a brown color appeared. A staining time should be adjusted according to staining intensity.

(12) Sealing a section: A resulting section was sealed with a sealing agent.

(13) Observation and analysis of results. A resulting section was observed under a light microscope, and staining results were recorded. Quantitative analysis could be performed using an image analysis software.

Statistical results of immunohistochemical data showed that lactylation levels of the NBS1 protein in tumor tissues of chemotherapy-resistant gastric cancer were significantly higher than those in tumor tissues of gastric cancer sensitive to chemotherapy (FIG. 6B). Therefore, the higher the lactylation level of the NBS1 protein, the poorer the chemotherapy efficacy for tumor patients.

In summary, inventors of this application discovered through lactylation proteomics that lysine (K) at position 388 of the NBS1 protein is the lactylated amino acid. In this application, it has been experimentally confirmed that lactylated NBS1 protein not only promotes MRN complex formation but also has multiple functions, such as enhancing tumor cell resistance to radiotherapy and chemotherapy and increasing homologous recombination (HR) in DNA repair. The lactylated NBS1 protein has the following potential applications: (1) It can regulate function of the NBS1 protein; (2) It can serve as a biomarker to predict efficacy of radiotherapy or chemotherapy in tumor patients; (3) Tumor resistance to radiotherapy or chemotherapy can be reversed by targeted inhibition of the lactylated NBS1 protein.

Lactylation of the NBS1 protein can serve as a predictive factor for prognosis and efficacy of neoadjuvant chemotherapy in tumor patients. Lactylation plays an important role in regulating the function of the NBS1 protein and in predicting the therapeutic efficacy for tumor patients.

The nucleotide sequence of plasmid PLVX-Flag-TIP60 in this application is as follows:

GGAAGGGCTAATTCACTCCCAAAGAAGACAAGATATCCTTGATCTGTGGATCTACCACACACAAGGCTACTTCCCTGATTAGCAGAACTACACACCAGGGCCAGGGGTCAGATATCCACTGACCTTTGGATGGTGCTACAAGCTAGTACCAGTTGAGCCAGATAAGGTAGAAGAGGCCAATAAAGGAGAGAACACCAGCTTGTTACACCCTGTGAGCCTGCATGGGATGGATGACCCGGAGAGAGAAGTGTTAGAGTGGAGGTTTGACAGCCGCCTAGCATTTCATCACGTGGCCCGAGAGCTGCATCCGGAGTACTTCAAGAACTGCTGATATCGAGCTTGCTACAAGGGACTTTCCGCTGGGGACTTTCCAGGGAGGCGTGGCCTGGGCGGGACTGGGGAGTGGCGAGCCCTCAGATCCTGCATATAAGCAGCTGCTTTTTGCCTGTACTGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGTGGCGCCCGAACAGGGACTTGAAAGCGAAAGGGAAACCAGAGGAGCTCTCTCGACGCAGGACTCGGCTTGCTGAAGCGCGCACGGCAAGAGGCGAGGGGCGGCGACTGGTGAGTACGCCAAAAATTTTGACTAGCGGAGGCTAGAAGGAGAGAGATGGGTGCGAGAGCGTCAGTATTAAGCGGGGGAGAATTAGATCGCGATGGGAAAAAATTCGGTTAAGGCCAGGGGGAAAGAAAAAATATAAATTAAAACATATAGTATGGGCAAGCAGGGAGCTAGAACGATTCGCAGTTAATCCTGGCCTGTTAGAAACATCAGAAGGCTGTAGACAAATACTGGGACAGCTACAACCATCCCTTCAGACAGGATCAGAAGAACTTAGATCATTATATAATACAGTAGCAACCCTCTATTGTGTGCATCAAAGGATAGAGATAAAAGACACCAAGGAAGCTTTAGACAAGATAGAGGAAGAGCAAAACAAAAGTAAGACCACCGCACAGCAAGCGGCCGGCCGCTGATCTTCAGACCTGGAGGAGGAGATATGAGGGACAATTGGAGAAGTGAATTATATAAATATAAAGTAGTAAAAATTGAACCATTAGGAGTAGCACCCACCAAGGCAAAGAGAAGAGTGGTGCAGAGAGAAAAAAGAGCAGTGGGAATAGGAGCTTTGTTCCTTGGGTTCTTGGGAGCAGCAGGAAGCACTATGGGCGCAGCGTCAATGACGCTGACGGTACAGGCCAGACAATTATTGTCTGGTATAGTGCAGCAGCAGAACAATTTGCTGAGGGCTATTGAGGCGCAACAGCATCTGTTGCAACTCACAGTCTGGGGCATCAAGCAGCTCCAGGCAAGAATCCTGGCTGTGGAAAGATACCTAAAGGATCAACAGCTCCTGGGGATTTGGGGTTGCTCTGGAAAACTCATTTGCACCACTGCTGTGCCTTGGAATGCTAGTTGGAGTAATAAATCTCTGGAACAGATTTGGAATCACACGACCTGGATGGAGTGGGACAGAGAAATTAACAATTACACAAGCTTAATACACTCCTTAATTGAAGAATCGCAAAACCAGCAAGAAAAGAATGAACAAGAATTATTGGAATTAGATAAATGGGCAAGTTTGTGGAATTGGTTTAACATAACAAATTGGCTGTGGTATATAAAATTATTCATAATGATAGTAGGAGGCTTGGTAGGTTTAAGAATAGTTTTTGCTGTACTTTCTATAGTGAATAGAGTTAGGCAGGGATATTCACCATTATCGTTTCAGACCCACCTCCCAACCCCGAGGGGACCCGACAGGCCCGAAGGAATAGAAGAAGAAGGTGGAGAGAGAGACAGAGACAGATCCATTCGATTAGTGAACGGATCTCGACGGTATCGCCTTTAAAAGAAAAGGGGGGATTGGGGGGTACAGTGCAGGGGAAAGAATAGTAGACATAATAGCAACAGACATACAAACTAAAGAATTACAAAAACAAATTACAAAAATTCAAAATTTTCGGGTTTATTACAGGGACAGCAGAGATCCAGTTTATCGATGAGGCCCTTTCGTCTTCACTCGAGTTTACTCCCTATCAGTGATAGAGAACGTATGTCGAGTTTACTCCCTATCAGTGATAGAGAACGATGTCGAGTTTACTCCCTATCAGTGATAGAGAACGTATGTCGAGTTTACTCCCTATCAGTGATAGAGAACGTATGTCGAGTTTACTCCCTATCAGTGATAGAGAACGTATGTCGAGTTTATCCCTATCAGTGATAGAGAACGTATGTCGAGTTTACTCCCTATCAGTGATAGAGAACGTATGTCGAGGTAGGCGTGTACGGTGGGAGGCCTATATAAGCAGAGCTCGTTTAGTGAACCGTCAGATCGCCTGGAGAAGGATCATGTTCCGGTGTGGAGGCCTGGC GCGGAGGTGGTGAGTCCGGTGCCCGGGGCGGGGCGGAGGGAGCCAGGGGAGGTGGGTAGAGCCCGAGGCCCCCCAGTAGCCGACCCTGGCGTCGCGCTGTCTCCCCAGGGGGAGATAATCGAGGGCTGCCGCCTACCCGTGCTGCGGCGGAACCAGGACAACGAAGATGAGTGGCCCCTGGCCGAGATCCTGAGCGTGAAGGACATCAGTGGCCGGAAGCTTTTCTACGTCCATTACATTGACTTCAACAAACGTCTGGATGAATGGGTGACGCATGAGCGGCTGGACCTAAAGAAGATCCAGTTCCCCAAGAAAGAGGCCAAGACCCCCACTAAGAACGGACTTCCTGGGTCCCGTCCTGGCTCTCCAGAGAGAGAGGTGCCGGCCTCGGCGCAGGCCAGCGGGAAGACCTTGCCAATCCCGGTCCAGATCACACTCCGCTTCAACCTGCCCAAGGAGCGGGAGGCCATTCCCGGTGGCGAGCCTGACCAGCCGCTCTCCTCCAGCTCCTGCCTGCAGCCCAACCACCGCTCAACGAAACGGAAGGTGGAGGTGGTTTCACCAGCAACTCCAGTGCCCAGC GAGACAGCCCCGGCCTCGGTTTTTCCCCAGAATGGAGCCGCCCGTAGGGCAGTGGCAGCCCAGCCAGGACGGAAGCGAAAATCGAATTGTTTGGGCACTGATGAGGACTCCCAGGACAGCTCTGATGGAATACCGTCAGCACCACGCATGACTGGCAGCCTGGTGTCTGATCGAAGCCACGACGACATCGTCACCCGGATGAAGAACATTGAGTGCATTGAGCTGGGCCGGCACCGCCTCAAGCCGTGGTACTTCTCCCCGTACCCACAGGAACTCACCACATTGCCTGTCCTCTACCTGTGCGAGTTCTGCCTCAAGTACGGCCGTAGTCTCAAGTGTCTTCAGCGTCATTTGACCAAGTGTGACCTACGACATCCTCCAGGCAATGAGATTTACCGCAAGGGCACCATCTCCTTCTTTGAGATTGATGGACGTAAGAACAAGAGTTATTCCCAGAACCTGTGTCTTTTGGCCAAGTGTTTCCTTGACCATAAGACACTGTACTATGACACAGACCCTTTCCTCTTCTACGTCATGACAGAGTATGACTGTAAGGGCTTCCACATCGTGGGCTACTTCTCCAAGGAGAAAGAATCAACGGAAGACTACAATGTGGCCTGCATCCTAACCCTGCCTCCCTACCAGCGCCGGGGCTACGGCAAGCTGCTGATCGAGTTCAGCTATGAACTCTCCAAAGTGGAAGGGAAAACAGGGACCCCTGAGAAGCCCCTCTCAGACCTTGGCCTCC TATCCTATCGAAGCTACTGGTCCCAGACCATCCTGGAGATCCTGATGGGGCTGAAGTCGGAGAGCGGGGAGAGGCCACAGATCACCATCAATGAGATTAGTGAAATCACCAGCATCAAGAAGGAGGATGTCATCTCCACTCTGCAGTACCTCAATCTCATCAACTACTACAAGGGCCAGTACATCCTCACACTGTCAGAGGACATCGTGGATGGCCATGAGCGGGCCATGCTCAAGCGGCTCCTGCGGATCGACTCCAAGTGTCTGCACTTCACTCCCAAGGACTGGAGCAAGAGGGGGAAGTGGTGAGAATTCTACCGGGTAGGGGAGGCGCTTTTCCCAAGGCAGTCTGGAGCATGCGCTTTAGCAGCCCCGCTGGGCACTTGGCGCTACACAAGTGGCCTCTGGCCTCGCACACATTCCACATCCACCGGTAGGCGCCAACCGGCTCCGTTCTTTGGTGGCCCCTTCGCGCCACCTTCTACTCCTCCCCTAGTCAGGAAGTTCCCCCCCGCCCCGCAGCTCGCGTCGTGCAGGACGTGACAAATGGAAGTAGCACGTCTCACTAGTCTCGTGCAGATGGACAGCACCGCTGAGCAATGGAAGCGGGTAGGCCTTTGGGGCAGCGGCCAATAGCAGCTTTGCTCCTTCGCTTTCTGGGCTCAGAGGCTGGGAAGGGGTGGGTCCGGGGGCGGGCTCAGGGGCGGGCTCAGGGGCGGGGCGGGCGCCCGAAGGTCCTCCGGAGGCCCGGCATTCTGCACGCTTCAAAAGCGCACGTCTGCCGCGCTGTTCTCCTCTTCCTCATCTCCGGGCCTTTCGACCTGCAGCCCAAGCTTACCATGACCGAGTACAAGCCCACGGTGCGCCTCGCCACCCGCGACGACGTCCCCAGGGCCGTACGCACCCTCGCCGCCGCGTTCGCCGACTACCCCGCCACGCGCCACACCGTCGATCCGGACCGCCACATCGAGCGGGTCACCGAGCTGCAAGAACTCTTCCTCACGCGCGTCGGGCTCGACATCGGCAAGGTGTGGGTCGCGGACGACGGCGCCGCGGTGGCGGTCTGGACCACGCCGGAGAGCGTCGAAGCGGGGGCGGTGTTCGCCGAGATCGGCCCGCGCATGGCCGAGTTGAGCGGTTCCCGGCTGGCCGCGCAGCAACAGATGGAAGGCCTCCTGGCGCCGCACCGGCCCAAGGAGCCCGCGTGGTTCCTGGCCACCGTCGGCGTCTCGCCCGACCACCAGGGCAAGGGTCTGGGCAGCGCCGTCGTGCTCCCCGGAGTGGAGGCGGCCGAGCGCGCCGGGGTGCCCGCCTTCCTGGAGACCTCCGCGCCCCGCAACCTCCCCTTCTACGAGCGGCTCGGCTTCACCGTCACCGCCGACGTCGAGGTGCCCGAAGGACCGCGCACCTGGTGCATGACCCGCAAGCCCGGTGCCTGACGGGCGCGTCTGGAACAATCAACCTCTGGATTACAAAATTTGTGAAAGATTGACTGGTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTATAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGGGCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGGGGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGACGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAGACGAGTCGGATCTCCCTTTGGGCCGCCTCCCCGCCTGGAATTAATTCTGCAGTCGAGACCTAGAAAAACATGGAGCAATCACAAGTAGCAATACAGCAGCTACCAATGCTGATTGTGCCTGGCTAGAAGCACAAGAGGAGGAGGAGGTGGGTTTTCCAGTCACACCTCAGGTACCTTTAAGACCAATGACTTACAAGGCAGCTGTAGATCTTAGCCACTTTTTAAAAGAAAAGAGGGGACTGGAAGGGCTAATTCACTCCCAACGAAGACAAGATATCCTTGATCTGTGGATCTACCACACACAAGGCTACTTCCCTGATTAGCAGAACTACACACCAGGGCCAGGGGTCAGATATCCACTGACCTTTGGATGGTGCTACAAGCTAGTACCAGTTGAGCCAGATAAGGTAGAAGAGGCCAATAAAGGAGAGAACACCAGCTTGTTACACCCTGTGAGCCTGCATGGGATGGATGACCCGGAGAGAGAAGTGTTAGAGTGGAGGTTTGACAGCCGCCTAGCATTTCATCACGTGGCCCGAGAGCTGCATCCGGAGTACTTCAAGAACTGCTGATATCGAGCTTGCTACAAGGGACTTTCCGCTGGGGACTTTCCAGGGAGGCGTGGCCTGGGCGGGACTGGGGAGTGGCGAGCCCTCAGATCCTGCATATAAGCAGCTGCTTTTTGCCTGTACTGGGTCTCTCTGGTTAGACCAGATCTGAGCCTGGGAGCTCTCTGGCTAACTAGGGAACCCACTGCTTAAGCCTCAATAAAGCTTGCCTTGAGTGCTTCAAGTAGTGTGTGCCCGTCTGTTGTGTGACTCTGGTAACTAGAGATCCCTCAGACCCTTTTAGTCAGTGTGGAAAATCTCTAGCAGTAGTAGTTCATGTCATCTTATTATTCAGTATTTATAACTTGCAAAGAAATGAATATCAGAGAGTGAGAGGCCTTGACATTGCTAGCGTTTACCGTCGACCTCTAGCTAGAGCTTGGCGTAATCATGGTCATAGCTGTTTCCTGTGTGAAATTGTTATCCGCTCACAATTCCACACAACATACGAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTACAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGCGTCAATACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTGACGTCGACGGATCGGGAGATCAACTTGTTTATTGCAGCTTATAATGGTTACAAATAAAGCAATAGCATCACAAATTTCACAAATAAAGCATTTTTTTCACTGCATTCTAGTTGTGGTTTGTCCAAACTCATCAATGTATCTTATCATGTCTGGATCAACTGGATAACTCAAGCTAACCAAAATCATCCCAAACTTCCCACCCCATACCCTATTACCACTGCCAATTACCTGTGGTTTCATTTACTCTAAACCTGTGATTCCTCTGAATTATTTTCATTTTAAAGAAATTGTATTTGTTAAATATGTACTACAAACTTAGTAGTT (SEQ ID NO: 5)

Finally, it should be noted that the above examples are only used to illustrate the technical solution of the present application and not to limit the scope of protection of the present application. Although the present application is described in detail with reference to the preferred examples, it should be understood by those of ordinary skill in the art that modifications or equivalent substitutions may be made to the technical solutions of the present application without departing from the substance and scope of the technical solutions of the present application.

1. A kit for predicting efficacy of radiotherapy or chemotherapy for a tumor, comprising a reagent for detecting a lactylation level of Nijmegen breakage syndrome 1 (NBS1) protein, wherein a lactylation site is lysine at position 388 of the NBS1 protein.

2. The kit of claim 1, wherein the lactylation level of the NBS1 protein is regulated by a lysine lactyltransferase.

3. The kit of claim 2, wherein the lysine lactyltransferase is Tat-interactive protein 60 (TIP60).

4. The kit of claim 1, wherein the higher the lactylation level of the NBS1 protein, the worse the efficacy of chemotherapy or/and radiotherapy in a tumor patient.

5. The kit of claim 1, wherein the reagent is an antibody against NBS1-K388la.

6. The kit of claim 1, wherein lactylation of the NBS1 protein is binding of a lactyl group to an amino group via an amide bond, and the amino group is located at a side chain end of the lysine at position 388 of the NBS1 protein.

7. A pharmaceutical composition for reversing tumor resistance to radiotherapy or chemotherapy, comprising an agent that specifically inhibits lactylation of NBS1 protein and a pharmaceutically acceptable carrier.

8. The pharmaceutical composition of claim 7, wherein the agent comprises a gene editing reagent that modifies a lactylation site to prevent lactylation of the NBS1 protein, or a reagent that inhibits expression or function of Tat-interactive protein 60 (TIP60).

9. The pharmaceutical composition of claim 7, wherein the agent is selected from the group consisting of a small molecule inhibitor, a nucleic acid, and a polypeptide.

10. The pharmaceutical composition of claim 8, wherein the agent is an antibody against NBS1-K388la, or a plasmid comprising a spacer DNA sequence, an extended DNA sequence, and a prime editing guide RNA (pegRNA).

11. The pharmaceutical composition of claim 10, wherein the spacer DNA sequence is set forth in SEQ ID NO: 3, and the extended DNA sequence is set forth in SEQ ID NO: 4.

12. The pharmaceutical composition of claim 7, wherein the agent is a plasmid pU6-pegRNA-GG-NBS1-K388R.

13. The pharmaceutical composition of claim 10, wherein the plasmid is a plasmid pU6-pegRNA-GG-NBS1-K388R.

14. The pharmaceutical composition of claim 9, wherein the agent comprises two siRNAs, wherein a first siRNA has a nucleotide sequence of ACGGAAGGUGGAGGUGGUU (SEQ ID NO: 1), and a second siRNA has a nucleotide sequence of AAGAAGAUCCAGUUCCCCAAGUU (SEQ ID NO: 2).

15. A method for reversing tumor resistance to radiotherapy or chemotherapy, comprising administering a therapeutically effective amount of the pharmaceutical composition of claim 7 to a tumor patient exhibiting tumor resistance to radiotherapy or chemotherapy.

16. A method for reversing tumor resistance to radiotherapy or chemotherapy, comprising administering a therapeutically effective amount of the pharmaceutical composition of claim 10 to a tumor patient exhibiting tumor resistance to radiotherapy or chemotherapy.

17. A method for reversing tumor resistance to radiotherapy or chemotherapy, comprising administering a therapeutically effective amount of the pharmaceutical composition of claim 14 to a tumor patient exhibiting tumor resistance to radiotherapy or chemotherapy.

18. A gastric cancer cell line AGS, comprising in its genome a mutant NBS1 gene, wherein the mutant NBS1 gene comprises a mutation when compared to wild-type NBS1 gene, wherein the mutation changes the codon for lysine at position 388 of a protein encoded by the wild-type NBS1 gene to a codon for an amino acid that cannot be lactylated.

19. The gastric cancer cell line AGS of claim 18, wherein the amino acid that cannot be lactylated is arginine.
